(19) United States
(12) Patent Application Publication
(10) Pub. No.: US 2009/0291051 A1
(43) Pub. Date: Nov. 26, 2009

(54) COMPOSITIONS HAVING A HEATING EFFECT

(75) Inventors: Jerome Senee, Lardy (FR); Delphine Compain, Paris (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'OREAL, Paris (FR)

(21) Appl. No.: 12/423,220

(22) Filed: Apr. 14, 2009

Related U.S. Application Data
(60) Provisional application No. 61/047,778, filed on Apr. 25, 2008.

(51) Int. Cl. A61K 8/23; A61K 8/19; A61K 8/30; A61P 17/00

(52) U.S. Cl. 424/59; 424/63; 424/682

(57) ABSTRACT

A method, including applying to a keratin substance a layer of a composition containing, in an anhydrous medium, at least one exothermic metal salt, and, optionally, one or more of the following:
a layer of a composition containing a monocarbonylated or polycarbonylated self-tanning agent;
a layer of a shaving composition and/or a depilatory composition;
a layer of a composition containing a dye, a pigment, or both; and
a layer of a composition containing an active ingredient.

### REFERENCE TO PRIOR APPLICATION

This application claims priority to U.S. provisional application 61/047,778, filed Apr. 25, 2008, incorporated herein by reference.

### FIELD OF THE INVENTION

The present invention relates to the uses of a composition comprising a metal salt, particularly in the cosmetic field.

Additional aspects and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### DETAILED DESCRIPTION OF THE PREFERRED EMBODIMENTS

The inventors have found that compositions comprising a relatively high percentage of metal salt(s) (for example 10 to 30%) in order to have a satisfactory heating effect may be used, alone or combined with other compositions, in various cosmetic applications to obtain particular cosmetic results or effects.

1/ Use of a Composition Having a Heating Effect with a Composition Containing a Self-Tanning Agent Such as Dihydroxyacetone

According to a first aspect, the invention relates to a method for colouring keratin substances that comprises applying to the keratin substances:
at least one layer of at least one “heating” composition comprising, in an anhydrous medium, at least one exothermic metal salt; and
at least one layer of at least one composition comprising at least one monocarbonylated or polycarbonylated self-tanning agent such as dihydroxyacetone (DHA).

The expression “keratin substances” is understood to mean the skin of the body or of the face, keratin fibres such as the hair or eyelashes, the nails, and in particular the skin.

Another subject of the invention is the use of a composition comprising, in an anhydrous medium, at least one exothermic metal salt, in combination with a composition comprising at least one monocarbonylated or polycarbonylated self-tanning agent such as dihydroxyacetone (DHA) to obtain an intense and/or homogeneous colour over the keratin substances.

Another subject of the invention is a kit comprising:
a composition comprising, in an anhydrous medium, at least one exothermic metal salt; and
a composition comprising at least one monocarbonylated or polycarbonylated self-tanning agent such as dihydroxyacetone (DHA).

This example and also all the examples that follow are given by way of illustration and non-limitingly with a view to making the features of the invention stand out better. Unless indicated otherwise, the quantities therein are given in % by weight.

### Composition Example Comprising an Exothermic Metal Salt

| | |
|---|---|
| PEG-8 | 53.19% |
| Glycerol | 5% |
| Citric acid | 1% |
| Hydrophobic silica (AEROSIL R974) | 2% |
| Hydrophilic silica (AEROSIL 200) | 1% |
| PEG-7 caprylic/capric glycerides | 2% |
| n-Octanoyl-5-salicylic acid | 0.3% |
| Fragrance | 0.3% |
| Cyclopentasiloxane/dimethiconol (Dow Corning 1501 | 15% |
| Fluid) (binder) | |
| Mixture of magnesium sulphate and water (98/2) | 20% |
| Orange-coloured jojoba beads (250-600 microns) | 0.2% |
| (Florabeads jojoba 28/60 Sunburst Orange from | |
| Floratech) | |
| Calcium pantothenate | 0.01% |
| Fragrance | 0.3% |

2/ Use of a Composition Having a Heating Effect to Improve Shaving

According to another aspect, one subject of the invention is a shaving and/or epilation method that comprises applying to the keratin substances:
at least one layer of at least one composition comprising, in an anhydrous medium, at least one exothermic metal salt; and
at least one layer of at least one shaving composition and/or a depilatory composition.

Another subject of the invention is the use of a composition comprising, in an anhydrous medium, at least one exothermic metal salt in combination with a shaving and/or depilatory composition for softening the hairs of the skin and/or for improving the shaving and/or the removal of the hairs of the skin.

Another subject of the invention is a shaving kit comprising:
a composition comprising, in an anhydrous medium, at least one exothermic metal salt;
a shaving composition or a depilatory composition; and
optionally at least one razor, especially a disposable razor.

The following example illustrates a heating composition comprising an exothermic salt that can be used in the above method:

| | |
|---|---|
| PEG-8 | 52.6% |
| Glycerol | 5% |
| Hydrophobic silica (AEROSIL R974) | 2% |
| Hydrophilic silica (AEROSIL 200) | 1% |
| PEG-7 caprylic/capric glycerides | 2% |
| Citric acid | 1% |
| Zinc gluconate | 0.1% |
| n-Octanoyl-5-salicylic acid | 0.1% |
| Fragrance | 0.2% |
| Cyclopentasiloxane/dimethiconol (Dow Corning 1501 | 15% |
| Fluid) (binder) | |
| Mixture of magnesium sulphate and water (98/2) | 20% |
| Polyethylene powder (Gotalene 120 colourless 23 | 0.75% |
| from DuPont) | |
| Blue-coloured polyethylene powder (Gotalene 120 | 0.25% |
| blue 23 from DuPont) | |

### 3/ Use of a Composition Having a Heating Effect for Reducing Wrinkles

According to one variant, one subject of the invention is a method for treating, smoothing and/or reducing the wrinkles and fine lines of the skin that comprises applying to the keratin substances at least one layer of at least one composition comprising, in an anhydrous medium, at least one exothermic metal salt. Another subject of the invention is the use of at least one exothermic metal salt, in a cosmetic composition comprising an anhydrous medium, as an agent for treating, smoothing and/or reducing the wrinkles and fine lines of the skin.

The following example illustrates a heating composition comprising an exothermic salt that can be used in the above method:

| | |
|---|---|
| PEG-8 | 59% |
| Glycerol | 5% |
| Hydrophobic silica (AEROSIL R974) | 2.5% |
| Hydrophilic silica (AEROSIL 200) | 1.5% |
| PEG-7 caprylic/capric glycerides | 2% |
| Cyclopentasiloxane/dimethiconol (Dow Corning 1501 | 10% |
| Fluid) (binder) | |
| Mixture of magnesium sulphate and water (98/2) | 20% |

### 4/ Use of a Composition Having a Heating Effect for Reducing the Appearance of Cellulite

According to one variant, one subject of the invention is a method for treating and/or reducing cellulite, in particular the appearance of cellulite, that comprises applying to the keratin substances at least one layer of at least one composition comprising, in an anhydrous medium, at least one exothermic metal salt.

Another subject of the invention is the use of at least one exothermic metal salt, in a composition comprising an anhydrous medium, as an agent for treating and/or reducing cellulite, in particular the appearance of cellulite.

By way of example, it is possible to use the following composition:

| | |
|---|---|
| PEG-8 | 52.6% |
| Glycerol | 5% |
| Hydrophobic silica (AEROSIL R974) | 2% |
| Hydrophilic silica (AEROSIL 200) | 1% |
| PEG-7 caprylic/capric glycerides | 2% |
| Citric acid | 1% |
| Zinc gluconate | 0.1% |
| n-Octanoyl-5-salicylic acid | 0.1% |
| Fragrance | 0.2% |
| Cyclopentasiloxane/dimethiconol (Dow Corning 1501 | 15% |
| Fluid) (binder) | |
| Mixture of magnesium sulphate and water (98/2) | 20% |
| Polyethylene powder (Gotalene 120 colourless 23 | 0.75% |
| from DuPont) | |
| Blue-coloured polyethylene powder (Gotalene 120 | 0.25% |
| blue 23 from DuPont) | |

### 5/ Use of a Composition Having a Heating Effect for Reducing the Appearance of Marks or the Inhomogeneity of the Skin

According to one variant, one subject of the invention is a method for camouflaging and/or reducing the appearance of marks or the inhomogeneity of the skin, comprising the application to the skin of a cosmetic composition comprising, in an anhydrous medium, at least one exothermic metal salt.

According to one embodiment, the heating composition is used in combination with a composition comprising dyes, in particular azo dyes, pigments and/or encapsulated pigments comprising a rupturable capsule.

Another subject of the invention is a method for reducing marks on the skin and/or the inhomogeneity of the skin that comprises applying to the skin:
at least one layer of at least one composition comprising, in an anhydrous medium, at least one exothermic metal salt; and
at least one layer of at least one composition comprising dyes, in particular azo dyes, pigments and/or encapsulated pigments comprising a rupturable capsule.

Another subject of the invention is a kit comprising:
at least one composition comprising, in an anhydrous medium, at least one exothermic metal salt; and
at least one composition comprising dyes, in particular azo dyes, pigments and/or encapsulated pigments comprising a rupturable capsule.

The following example illustrates a kit comprising compositions that can be used in the above method:

### Composition 1 Comprising an Exothermic Salt

| | |
|---|---|
| PEG-8 | 46.5% |
| Glycerol | 5% |
| Hydrophobic silica (AEROSIL R974) | 2% |
| Hydrophilic silica (AEROSIL 200) | 1% |
| PEG-7 caprylic/capric glycerides | 2% |
| Citric acid | |
| n-Octanoyl-5-salicylic acid | |
| Fragrance | 0.3% |
| Cyclopentasiloxane/dimethiconol (Dow Corning 1501 | 15% |
| Fluid) (binder) | |
| Mixture of magnesium sulphate and water (98/2) | 14.7% |
| Red-coloured polyethylene beads (850 microns) | 2% |
| (Cirebelle 104 Crimson Spice from Sasol) | |

### Composition 2 Comprising Azo Dyes

| | |
|---|---|
| Menthol | 0.10% |
| Sodium citrate | 0.02% |
| Caffeine | 0.10% |
| Ethyl menthane carboxamide | 0.10% |
| Mentha piperita | 0.20% |
| CI 19140 | 0.03% |
| CI 61570 | 0.01% |
| CI 15510 | 0.03% |
| CI 17200 | 0.02% |
| Fragrance | 0.40% |
| Sodium Hyaluronate | 0.05% |
| Sodium Polyacrylate | 0.30% |
| Acrylates/C10-30 Alkyl Acrylate Crosspolymer | 0.40% |
| Bis-Peg-18 Methyl ether dimethyl silane | 3% |
| Butylene glycol | 3% |
| Ethanol | 20% |
| Water | 63.45% |
| Glycerol | 7% |
| PEG-60 Hydrogenated castor oil | 0.50% |
| PEG-7 Glyceryl cocoate | 1% |
| Panthenol | 0.25% |
| Ascorbyl glucoside | 0.05% |

### Composition 2′ Comprising Encapsulated Pigments

| | |
|---|---|
| Disodium EDTA | 0.10% |
| Pentasodium ethylenediamine tetramethylene | 0.10% |
| phosphonate | |
| Capryloyl salicylic acid | 0.30% |
| Triethanolamine | 0.75% |
| Calcium pantetheine sulphonate | 0.10% |
| Hydroxyapatite | 0.10% |
| Hydrolysed soy protein | 0.50% |
| Acrylates copolymer | 0.20% |
| Butyrospermum parkii | 3% |
| Petrolatum | 4% |
| Myristyl myristate | 3% |
| CI 77492 (and) CI77891 (and) Boron Nitride (and) | 0.70% |
| Acrylates/Ammonium methacrylate copolymer | |
| CI 77491 (and) CI77891 (and) Boron Nitride (and) | 0.30% |
| Acrylates/Ammonium methacrylate copolymer | |
| CI 77499 (and) CI77891 (and) Boron Nitride (and) | 0.01% |
| Acrylates/Ammonium methacrylate copolymer | |
| Phenoxyethanol | 0.50% |
| Collagen | 1% |
| Ethylhexyl salicylate | 5% |
| Octocrylene | 7% |
| Butyl methoxy dibenzoyl methane | 3% |
| Mica (and) CI77891 | 0.20% |
| Mica (and) CI77891 | 0.20% |
| Fragrance | 0.30% |
| Xanthan gum | 0.20% |
| Carbomer | 0.60% |
| Poly C10-30 alkyl acrylate | 1% |
| Cyclopentasiloxane | 10% |
| Aqua | 48.33% |
| Glycerol | 7% |
| Capryl glycol | 0.50% |
| Glyceryl stearate (and) PEG-100 stearate | 2% |
| Ascorbyl glucoside | 0.01% |

### 6/ Use of a Composition Having a Heating Effect for Reducing the Adhesion of Microorganisms

According to another aspect, one subject of the invention is a method for preventing and/or reducing the adhesion of microorganisms to the skin and/or the mucous membranes, comprising the application to the skin of a composition comprising, in an anhydrous medium, at least one exothermic metal salt.

Another subject of the invention is the use of at least one exothermic metal salt, in a composition comprising an anhydrous medium, as an agent for preventing and/or reducing the adhesion of microorganisms to the skin and/or mucous membranes.

The following example illustrates a composition that can be used in the above method:

| | |
|---|---|
| PEG-8 | 52.6% |
| Glycerol | 5% |
| Hydrophobic silica (AEROSIL R974) | 2% |
| Hydrophilic silica (AEROSIL 200) | 1% |
| PEG-7 caprylic/capric glycerides | 2% |
| Citric acid | 1% |
| Zinc gluconate | 0.1% |
| n-Octanoyl-5-salicylic acid | 0.1% |
| Fragrance | 0.2% |
| Cyclopentasiloxane/dimethiconol (Dow Corning 1501 | 15% |
| Fluid) (binder) | |
| Mixture of magnesium sulphate and water (98/2) | 20% |
| Polyethylene powder (Gotalene 120 colourless 23 | 0.75% |
| from DuPont) | |
| Blue-coloured polyethylene powder (Gotalene 120 | 0.25% |
| blue 23 from DuPont) | |

### 8/ Use of a Composition Having a Heating Effect on the Eye Contour Area (Dark Circles and Bags) and of a Composition Containing an Active Ingredient

Another subject of the invention is a cosmetic method for treating and/or reducing bags and/or dark circles under the eyes that comprises applying to the bags and/or dark circles:
at least one layer of a composition comprising, in an anhydrous medium, at least one exothermic metal salt; and
at least one layer of a composition comprising at least one active ingredient chosen from caffeine and derivatives thereof, caffeine citrate, theophylline and derivatives thereof, theobromine, acefylline, aminophylline, chloroethyltheophylline, diprofylline, diniprophylline, etamiphylline and derivatives thereof, etofylline, proxyphylline, ephedrine and derivatives thereof, combinations of caffeine and silanol, compounds of natural origin containing xanthic bases such as extracts of tea, of coffee, of guarana, of cocoa, of mate, of cola (Cola nitida), a maritime pine bark extract such as Pycnogénol® from Biolandes, manganese gluconate (Givobio GMn® from Seppic), an extract of Ammi visnaga such as Visnadine from Indena, lupin extract (Eclaline® from Silab), the protein coupling of hydrolysed wheat/palmitic acid with palmitic acid such as Epaline 100 from Laboratoires Carilène, the extract of a bitter orange blossom (Remoduline® from Silab), vitamin P and derivatives thereof such as methyl-4-esculetol sodium monoethanoate sold under the name Permethol® by Sephytal, extracts of Ruscus, of horse chestnut, of ivy, of ginseng and of sweet clover, nicotinate and derivatives thereof, lysine and derivatives thereof such as Asparlyne® from Solabia, an extract of black tea such as Kombuchka from Sederma, rutin salts, a Corallina officinalis algae extract such as that sold by CODIF, and mixtures thereof.

Another subject of the invention is the use of a composition comprising, in an anhydrous medium, at least one exothermic metal salt, in combination with a composition comprising at least one active ingredient chosen from caffeine and derivatives thereof, caffeine citrate, theophylline and derivatives thereof, theobromine, acefylline, aminophylline, chloroethyltheophylline, diprofylline, diniprophylline, etamiphylline and derivatives thereof, etofylline, proxyphylline, ephedrine and derivatives thereof, combinations of caffeine and silanol, compounds of natural origin containing xanthic bases such as extracts of tea, of coffee, of guarana, of cocoa, of mate, of cola (Cola nitida), a maritime pine bark extract such as Pycnogénol from Biolandes, manganese gluconate (Givobio GMn® from Seppic), an extract of Ammi visnaga such as Visnadine from Indena, lupin extract (Eclaline® from Silab), the protein coupling of hydrolysed wheat/palmitic acid with palmitic acid such as Epaline 100 from Laboratoires Carilène, the extract of a bitter orange blossom (Remoduline® from Silab), vitamin P and derivatives thereof such as methyl-4-esculetol sodium monoethanoate sold under the name Permethol® by Sephytal, extracts of Ruscus, of horse chestnut, of ivy, of ginseng and of sweet clover, nicotinate and derivatives thereof, lysine and derivatives thereof such as Asparlyne® from Solabia, an extract of black tea such as Kombuchka from Sederma, rutin salts, a Corallina officinalis algae extract such as that sold by CODIF, and mixtures thereof, for treating and/or reducing bags and/or dark circles under the eyes.

Another subject of the invention is a kit comprising:
a composition comprising, in an anhydrous medium, at least one exothermic metal salt; and
a composition comprising at least one active ingredient chosen from caffeine and derivatives thereof, caffeine citrate, theophylline and derivatives thereof, theobromine, acefylline, aminophylline, chloroethyltheophylline, diprofylline, diniprophylline, etamiphylline and derivatives thereof, etofylline, proxyphylline, ephedrine and derivatives thereof, combinations of caffeine and silanol, compounds of natural origin containing xanthic bases such as extracts of tea, of coffee, of guarana, of cocoa, of mate, of cola (Cola nitida), a maritime pine bark extract such as Pycnogénol from Biolandes, manganese gluconate (Givobio GMn® from Seppic), an extract of Ammi visnaga such as Visnadine from Indena, lupin extract (Eclaline® from Silab), the protein coupling of hydrolysed wheat/palmitic acid with palmitic acid such as Epaline 100 from Laboratoires Carilene, the extract of a bitter orange blossom (Remoduline® from Silab), vitamin P and derivatives thereof such as methyl-4-esculetol sodium monoethanoate sold under the name Permethol® by Sephytal, extracts of Ruscus, of horse chestnut, of ivy, of ginseng and of sweet clover, nicotinate and derivatives thereof, lysine and derivatives thereof such as Asparlyn® from Solabia, an extract of black tea such as Kombuchka from Sederma, rutin salts, a Corallina officinalis algae extract such as that sold by CODIF, and mixtures thereof;
and mixtures thereof.

The following example illustrates a kit comprising compositions that can be used in the above method:

### Composition 1 Comprising a Metal Salt

| | |
|---|---|
| PEG-8 | 50% |
| Glycerol | 22% |
| Hydrophobic silica (AEROSIL R974) | 3% |
| Hydrophilic silica (AEROSIL 200) | 2% |
| PEG-7 caprylic/capric glycerides | 2% |
| Citric acid | 0.5% |
| n-Octanoyl-5-salicylic acid | 0.1% |
| Fragrance | 0.2% |
| Mixture of magnesium sulphate and water (98/2) | 20% |
| Sodium hyaluronate | 0.01% |
| Red-coloured polyethylene powder (PFL RED 7044 | 0.2% |
| from Toshiki Pigments) | |

### Composition 2 Comprising Caffeine

| | |
|---|---|
| Mixture of magnesium sulphate and water (98/2) | 1.75% |
| Caffeine | 0.20% |
| Pentaerythrityl tetraisostearate (and) silica | 0.20% |
| dimethyl silylate | |
| Silica microspheres (SB 700 from Myoshi Kasei) | 2.50% |
| Tristearin (and) acetylated glycol stearate | 1% |
| Phenoxyethanol | 0.50% |
| CI 77891 (and) MICA | 0.50% |
| CI 77891 (and) MICA | 2.50% |
| Cyclohexasiloxane | 15% |
| Cetyl PEG/PPG-10/1 Dimethicone | 1.50% |
| Stearyl dimethicone | 2% |
| Dimethicone (and) Dimethiconol | 5% |
| Methylsilanol/Silicate crosspolymer | 1.50% |
| Ethanol | 6% |
| Water | 54.15% |
| Glycerol | 5% |
| Polyglyceryl-4-Isostearate | 0.50% |
| Tocopheryl acetate | 0.20% |

### Composition Comprising a Metal Salt (“Heating” Composition)

The composition comprising at least one metal salt employed in the various uses, kits and methods cited above is exothermic, that is to say that it has a heating effect, and therefore that the user experiences a warming sensation during application of said composition to wet or dampened skin. It is a composition for which the temperature, in the presence of water (water added during use or else water present in the skin), may rise by several degrees (one to twenty degrees) instantaneously.

This composition according to the invention is constituted of an anhydrous medium. The term “anhydrous” is understood here to mean an almost anhydrous medium, that is to say that generally comprises less than 6% by weight of water, preferably less than 4% by weight of water, and better still less than 1% by weight of water relative to the total weight of the composition. It is preferably completely anhydrous and therefore free of water.

Since this composition is preferably a cosmetic composition and is therefore preferably intended for topical application, it preferably comprises a physiologically acceptable medium, that is to say one that is compatible with the skin, mucous membranes and/or keratin fibres.

### a) Exothermic Metal Salts

The metal salts used in the present invention are salts having exothermic properties, that is to say that release heat when they are dampened. They are chosen, in particular, from alkaline-earth metal salts, magnesium salts and mixtures thereof.

As alkaline-earth metal salts, mention may in particular be made of calcium salts and, more especially, calcium halides such as calcium iodide, chloride and bromide. It is preferably calcium chloride.

As magnesium salts, mention may especially be made of magnesium sulphate.

It is also possible to use a mixture of several salts.

According to one preferred embodiment of the invention, the exothermic metal salt is chosen from magnesium sulphate, calcium chloride and mixtures thereof.

According to one embodiment, the amount of metal salt(s) is advantageously greater than or equal to 10% by weight, preferably greater than or equal to 15% by weight relative to the total weight of the composition.

The amount of metal salt(s) may range, for example, from 10 to 50% by weight, preferably from 15 to 40% by weight and better still from 15 to 25% by weight relative to the total weight of the composition.

### b) Hydrophilic Silica

According to one embodiment, the composition comprising a metal salt comprises one or more hydrophilic silicas. The amount of hydrophilic silica(s) may range, for example, from 0.1 to 20% by weight, preferably from 0.5 to 15% by weight and better still from 1 to 10% by weight relative to the total weight of the composition.

The expression “hydrophilic silica” is understood in the present application to mean not only pure hydrophilic silicas but also particles coated with hydrophilic silica.

The hydrophilic silicas that can be used in the composition of the invention are preferably amorphous, and they may be pyrogenic or precipitated in origin. They are generally in powder form.

Fumed (pyrogenic) silicas are obtained by continuous flame pyrolysis, at 1000° C., of silicon tetrachloride (SiCl₄) in the presence of hydrogen and oxygen.

Precipitated silicas are obtained by reaction of an acid over solutions of alkali metal silicates, preferably sodium silicate.

According to one preferred embodiment of the invention, the hydrophilic silica is selected from silicas having a specific surface area of 30 to 500 m²/g, and a number-average particle size ranging from 3 to 50 nm. These are more particularly the hydrophilic silicas described in Tables (1) and (2) below, and mixtures thereof.

TABLE (1)

| | Trade name | | | |
|---|---|---|---|---|
| | | AEROSIL | AEROSIL | AEROSIL |
| | AEROSIL 90 | 130 | 150 | 200 |
| | (Degussa- | (Degussa- | (Degussa- | (Degussa- |
| | Hüls) | Hüls) | Hüls) | Hüls) |
| Production | Pyrolysis | Pyrolysis | Pyrolysis | Pyrolysis |
| method | | | | |
| BET surface | 90 ± 15 | 130 ± 25 | 150 ± 15 | 200 ± 15 |
| area (m²/g) | | | | |
| Average | 20 | 16 | 14 | 12 |
| particle | | | | |
| size (nm) | | | | |
| Observation | | | | aggregate |
| | | | | size: |
| | | | | 10-30 μm and |
| | | | | 200 μm |

TABLE 2

| | Trade name | | | |
|---|---|---|---|---|
| | | AEROSIL | | SILICA FK |
| | AEROSIL 300 | 380 | AEROSIL OX | 320 DS |
| | (Degussa- | (Degussa- | 50 (Degussa- | (Degussa- |
| | Hüls) | Hüls) | Hüls) | Hüls) |
| Production | Pyrolysis | Pyrolysis | Pyrolysis | Pyrolysis |
| method | | | | |
| BET surface | 300 ± 30 | 380 ± 30 | 50 ± 25 | 170 ± 25 |
| area (m²/g) | | | | |
| Average | 7 | 7 | 40 | 18 |
| particle | | | | |
| size (nm) | | | | |

The hydrophilic silica used in the composition of the invention may also be composed of a particle covered wholly or partly with silica, in particular an inorganic particle covered wholly or partly with silica, such as silica beads containing titanium oxide that are sold under the name TORAYCERAM S-IT® by Toray; silica/alumina microspheres containing titanium oxide (size: 105 μl) that are sold under the name Z-LIGHT-SPHERE W 1012® by Zeelan; the amorphous precipitated synthetic silica/titanium oxide particles (size: 106-500μ) that are sold under the name NEOSIL PC20S® by Crosfield; the nylon-6/silica/titanium oxide fibres (length of 2 mm and thickness of 2 denier) that are sold under the name FIBERLON Y2® by Wackherr; the silica that is coated with titanium dioxide and covered with porous silica (85/5/10) (size: 0.6 p) that is sold under the name ACS-0050510® by SACI-CFPA; the anatase titanium oxide nanomaterial treated with alumina and silica, at 40% in water (size: 60 nm, monodisperse), that is sold under the name MIRASUN TIW 60® by Rhodia Chimie CRA; the anatase titanium oxide nanomaterial (60 nm) coated with 15/5/3 silica/alumina/cerium IV in aqueous dispersion at 32% that is sold under the name MIRASUN TIW 160® by Rhodia Chimie CRA; the anatase titanium oxide nanomaterial treated with alumina and silica (34/4.3/1.7) in aqueous dispersion at 40% that is sold under the name TIOVEIL AQ-N® by Uniqema; the titanium oxide nanomaterial coated with silica (66/33) (particle size of the titanium dioxide: 30 nm; thickness of silica: 4 nm) that is sold under the name MAXLIGHT TS-04® by Nichimen Europe PLC; and the titanium oxide nanomaterial coated with silica (80/20) (particle size of the titanium dioxide: 30 nm; thickness of silica: 2 nm) that is sold under the name MAXLIGHT TS-042® by Nichimen Europe PLC.

As hydrophilic silica it is preferred to use fumed silicas and especially those sold under the name AEROSIL (INCI name: silica) and especially that sold under the name AEROSIL 200 by Degussa-Huls.

### c) Hydrophobic Silica

The composition comprising a metal salt may comprise one or more hydrophobic silicas. The amount of hydrophobic silica(s) may range, for example, from 0.1 to 20% by weight, preferably from 0.5 to 15% by weight and better still from 5 to 15% by weight relative to the total weight of the composition.

The expression “hydrophobic silica” is understood in the present application to mean not only pure hydrophobic silicas but also particles coated with hydrophobic silica.

The hydrophobic silicas that can be used in the composition of the invention are preferably amorphous and pyrogenic in origin. They are preferably in powder form.

The amorphous hydrophobic silicas of pyrogenic origin are obtained from hydrophilic silicas. The latter are obtained by continuous flame pyrolysis, at 1000® C., of silicon tetrachloride (SiCl₄) in the presence of hydrogen and oxygen. They are then made hydrophobic by treatment with halogenated silanes, alkoxysilanes or silazanes. The hydrophobic silicas differ from the hydrophilic starting silicas in qualities including a lower density of silanol groups and a smaller level of water vapour adsorption.

According to one preferred embodiment of the invention, the hydrophobic silica is selected from silicas having a specific surface area of 50 to 500 m²/g and a number-average particle size of from 3 to 50 nm. These are, more particularly, the hydrophobic silicas described in Table (3) below, and mixtures thereof.

TABLE 3

| | Trade name | | | | |
|---|---|---|---|---|---|
| | AEROSIL | AEROSIL | AEROSIL | AEROSIL | AEROSIL |
| | R202 | R805 | R812 | R972 | R974 |
| | (Degussa- | (Degussa- | (Degussa- | (Degussa- | (Degussa- |
| | Hüls) | Hüls) | Hüls) | Hüls) | Hüls) |
| BET | 90 ± 20 | 150 ± 25 | 260 ± 30 | 110 ± 20 | 170 ± 20 |
| surface | | | | | |
| area | | | | | |
| (m²/g) | | | | | |
| Average | 14 | 12 | 7 | 16 | 12 |
| particle | | | | | |
| size (nm) | | | | | |

The hydrophobic silica used in the composition of the invention may also be composed of a particle covered wholly or partly with silica, in particular an inorganic particle covered wholly or partly with hydrophobic silica, such as pigments and metal oxides covered with hydrophobic silica. These particles may also have optical effects in the product and also on the skin; for example, they may have a mattifying or slightly whitening effect.

As hydrophobic silica it is preferred to use a hydrophobic fumed silica which is surface-treated with a dimethylsiloxane, such as that sold under the name AEROSIL R974 (INCI name: Silica Dimethyl Silylate) by Degussa-Hüls.

According to one embodiment, the composition comprising a metal salt comprises at least one hydrophilic silica and at least one hydrophobic silica, as described above.

### d) Polyols

According to one preferred embodiment of the invention, the composition comprising a metal salt also contains at least one polyol since polyols complement the exothermic action of the salts.

As polyols, mention may especially be made of polyols having at least 2 hydroxyl groups and at least 3 carbon atoms, such as glycerol, diglycerol, and glycols such as propylene glycol, dipropylene glycol, butylene glycol, hexylene glycol, polyethylene glycol and polyethylene glycols having a molecular weight of less than 600 such as PEG-8 (or polyethylene 400), sugars such as sorbitol, and mixtures thereof.

As polyols, use is preferably made of glycerol, butylene glycol, propylene glycol, dipropylene glycol, PEG-8 and mixtures thereof.

The amount of polyol(s) depends on the amount of salts present in the composition and the desired heating effect. It may range, for example, from 0.5 to 80% by weight, preferably from 5 to 75% by weight, better still from 10 to 70% by weight and better still from 20 to 65% by weight and better still from 30 to 60% by weight relative to the total weight of the composition comprising a metal salt.

### e) Anhydrous Medium

The composition comprising a metal salt according to the invention is preferably composed of an oily medium comprising at least one oil. The amount of oil(s) may range, for example, from 5 to 80% by weight, preferably from 10 to 60% by weight, better still from 10 to 40% by weight relative to the total weight of the composition.

The term “oil” is understood to mean a fatty substance that is liquid at ambient temperature (25° C.) and atmospheric pressure.

As oils that can be used in the composition of the invention, mention may be made, for example, of:
hydrocarbon oils of plant origin, such as liquid triglycerides of fatty acids comprising from 4 to 10 carbon atoms, such as triglycerides of heptanoic acid or octanoic acid, or else, for example, sweet almond, sunflower, maize, soybean, pumpkin, coriander, grape seed, sesame, hazelnut, apricot (Prunus armenica), macadamia, arara, castor or avocado oils, triglycerides of caprylic/capric acids, such as those sold by Stearineries Dubois or those sold under the names Miglyol 810, 812 and 818 by Dynamit Nobel, jojoba oil or shea butter oil;
synthetic esters and ethers, in particular of fatty acids, such as oils of formulae R¹COOR² and R¹OR² in which R¹ represents the residue of a fatty acid comprising from 8 to 29 carbon atoms and R² represents a branched or unbranched hydrocarbon chain comprising from 3 to 30 carbon atoms, such as, for example, purcellin oil or 660084 PCL-LIQUID from SYMRISE (mixture of cetylstearyl 2-ethylhexanoate and isopropyl myristate), isononyl isononanoate, isopropyl myristate, isopropyl palmitate, 2-ethylhexyl palmitate (or octyl palmitate), 2-octyldodecyl stearate, 2-octyldodecyl erucate or isostearyl isostearate; hydroxylated esters, such as isostearyl lactate, octyl hydroxystearate, octyldodecyl hydroxystearate, diisostearyl malate, triisocetyl citrate or heptanoates, octanoates or decanoates of fatty alcohols; polyol esters, such as propylene glycol dioctanoate, neopentyl glycol diheptanoate and diethylene glycol diisononanoate; and pentaerythritol esters, such as pentaerythrityl tetraisostearate;
linear or branched hydrocarbons of mineral or synthetic origin, such as volatile or non-volatile liquid paraffins and their derivatives, liquid petrolatum, polydecenes or hydrogenated polyisobutene, such as Parleam® oil;
fatty alcohols having from 8 to 26 carbon atoms, such as cetyl alcohol, stearyl alcohol and their mixture (cetearyl alcohol), octyldodecanol, 2-butyloctanol, 2-hexyldecanol, 2-undecylpentadecanol, oleyl alcohol or linoleyl alcohol;
fluorinated oils which partially comprise hydrocarbon and/or silicone, such as those described in the document JP-A-2-295912. Mention may also be made, as fluorinated oils, of perfluoromethylcyclopentane and perfluoro-1,3-dimethylcyclohexane, sold under the names of “FLUTEC PCl®” and “FLUTEC PC3®” by BNFL Fluorochemicals; perfluoro-1,2-dimethylcyclobutane; perfluoroalkanes, such as dodecafluoropentane and tetradecafluorohexane, sold under the names of “PF 5050®” and “PF 5060®” by 3M, or bromoperfluorooctyl, sold under the name of “FORALKYL®” by Atochem; nonafluoromethoxybutane sold under the name “MSX 4518” by 3M and nonafluoroethoxyisobutane; perfluoromorpholine derivatives such as 4-trifluoromethylperfluoromorpholine sold under the name “PF 5052®” by 3M;
silicone oils, such as volatile or non-volatile polydimethylsiloxanes (PDMSs) comprising a linear or cyclic silicone chain which are liquid or pasty at ambient temperature, in particular cyclopolydimethylsiloxanes (cyclomethicones), such as cyclohexasiloxane and cyclopentasiloxane; polydimethylsiloxanes comprising pendant alkyl, alkoxy or phenyl groups or alkyl, alkoxy or phenyl groups at the end of the silicone chain, which groups have from 2 to 24 carbon atoms; phenylated silicones, such as phenyltrimethicones, phenyldimethicones, phenyl(trimethylsiloxy)diphenylsiloxanes, diphenyldimethicones, diphenyl(methyldiphenyl)trisiloxanes, (2-phenylethyl)trimethylsiloxysilicates and polymethylphenylsiloxanes;
and their mixtures.

The term “hydrocarbon oil” is understood to mean, in the list of the oils mentioned above, any oil comprising predominantly carbon and hydrogen atoms and optionally ester, ether, fluorinated, carboxylic acid and/or alcohol groups.

The composition comprising a metal salt may comprise at least one makeup-removing oil, to which it is possible to add one or more other makeup-removing or non-makeup-removing oils. The makeup-removing oils may be chosen, in particular, from branched hydrocarbons of mineral origin such as hydrogenated polyisobutene, the fatty acid esters described above, and mixtures thereof. As fatty acid esters that can be used as makeup-removing oils, mention may more particularly be made of ethylhexyl palmitate, ethylhexyl stearate, isopropyl myristate, isopropyl palmitate, isobutyl palmitate, pentaerythritol caprate/caprylate, cetearyl isononanoate, isodecyl isononanoate, isononyl isononanoate, isotridecyl isononanoate, 2-ethylhexyl caprate/caprylate, and mixtures thereof.

In addition to the oils indicated above, the composition comprising a metal salt may contain other fatty substances, such as fatty acids comprising from 8 to 30 carbon atoms, such as stearic acid; silicone resins such as trifluoromethyl(C₁₋₄)alkyldimethicone and trifluoropropyldimethicone; silicone gums (INCI name: Dimethiconol) alone or as a mixture with a silicone oil, such as the product sold by Dow Corning under the name Dow Corning 1501 Fluid, which is a mixture of dimethiconol and of cyclopentasiloxane in a proportion of 14.7/85.3 (dimethiconol); silicone elastomers such as those sold under the names KSG by Shin-Etsu; waxes, for example mineral waxes, waxes of animal origin such as beeswax, waxes of plant origin, hydrogenated oils that are solid at 25° C., fatty esters and glycerides that are solid at 25° C., synthetic waxes such as polymethylene wax, silicone waxes; and mixtures thereof.

According to one embodiment, the composition comprising the exothermic metal salt may also comprise at least one foaming surfactant.

### Additives

The composition may, in addition, contain one or more other additives, especially lipophilic additives, including those conventionally used in cosmetic compositions, in particular in cleansing or makeup-removing compositions.

Mention may especially be made of exothermic compounds other than salts and polyols, such as for example zeolites which are silico-aluminates. As zeolites, mention may especially be made of activated zeolites, and for example zeolites A, zeolites X such as those sold by Fluka and Union Carbide, zeolites MAP such as described in document EP-A-384070, activated zeolites A such as described in document EP-A-187912 and especially those sold under the name ADVERA 401N and ADVERA 402N by PQ Corporation.

Mention may also be made, as additives, of: surfactants; fragrances; preservatives, antioxidants; sequestrants; fillers; dyes; cosmetic or dermatological active ingredients; or mixtures thereof. These adjuvants are used in the customary proportions for cleansing and/or care compositions, for example from 0.01 to 10% of the total weight of the composition. These adjuvants must be of a nature and used in an amount such that they do not disrupt the properties desired for the composition of the invention.

According to one particular embodiment of the invention, the composition comprising a metal salt contains, as fillers, exfoliating particles that will enable the skin to be scrubbed. As exfoliating particles, it is possible to use exfoliating or scrubbing particles of mineral, plant or organic origin. Thus, it is possible to use, for example, polyethylene beads or powder, such as those sold under the name Microthene MN 727 or Microthene MN 710-20 by Equistar or such as the powder sold under the name Gotalene 120 colourless 2 by DuPont; nylon particles such as those sold by Arkema under the name Orgasol 2002 EXD NAT COS; fibres such as polyamide fibres, such as those sold by Utexbel under the name PULPE POLYAMIDE 12185 TAILLE 0.3 MM (Polyamide Pulp 12185 Size 0.3 mm); polyvinyl chloride powder; pumice (INCI name: pumice) such as the pumice 3/B from Eyraud; the ground shells of fruit stones such as apricot kernel or nutshell homogenate; sawdust; glass beads; alumina (aluminium oxide) (INCI name: Alumina) such as the product sold under the name Dermagrain 900 by Marketech International; sugar crystals; beads which melt on application to the skin, such as for example spheres based on mannitol and cellulose sold under the name Unispheres by Induchem, agar-based capsules sold under the name Primasponge by Cognis, and spheres based on jojoba esters sold under the name Floraspheres by Floratech; and mixtures thereof.

The composition comprising a metal salt may also contain other fillers such as, for example, talc, modified or unmodified starch, especially starches esterified with octenylsuccinic anhydride and more particularly aluminium starch octenyl succinate such as the product sold by National Starch under the name Dry-Flo.

These fillers (exfoliating and other fillers) may be present in an amount ranging, for example, from 0.5 to 20% by weight, preferably from 1 to 15% by weight, better still from 1 to 10% and better still from 2 to 5% by weight relative to the total weight of the composition.

The examples above are given by way of illustration and non-limitingly with a view to making the features of the invention stand out better. The amounts thereof are given in % by weight unless otherwise stated.

The composition comprising a metal salt of the invention is not limited in form and may be in the form of a gel. It may be in the form of a spray or an aerosol, a patch, a pad, an anhydrous film or a wipe. It may be used with a supply of water that comes from wet skin or from another composition comprising an aqueous phase.

During use, it is possible to apply the composition to the skin (especially the skin of the face), preferably as a thick layer, then the hands are dampened and the face is massaged with the wet hands. It is optionally possible to redampen the hands and again massage the face. It finishes with rinsing the face. Another method comprises first dampening the skin, then applying the composition to the wet skin and massaging the face, possibly or possibly not after having dampened the hands.

In the present invention where two or more layers are used, they can be applied in any order. Where water is used in combination with an invention layer or layers, it can be present before, added during, or added after application of the layer(s), including any or all of these permutations. Preferably, the layer of at least one composition comprising, in an anhydrous medium, at least one exothermic metal salt is applied as the first layer.

In the invention, the various layers described herein can be applied more than once, and in any order.

While the claims below focus on the methods described herein, the various compositions described, and their combinations, also make up a part of the invention.

As used herein, the words “a” and “an” and the like carry the meaning of “one or more.”

The above written description of the invention provides a manner and process of making and using it such that any person skilled in this art is enabled to make and use the same, this enablement being provided in particular for the subject matter of the appended claims, which make up a part of the original description.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A method, comprising applying to a keratin substance a layer of a composition comprising, in an anhydrous medium, at least one exothermic metal salt, and, optionally, one or more of the following:
a layer of a composition comprising a monocarbonylated or polycarbonylated self-tanning agent;
a layer of a shaving composition and/or a depilatory composition;
a layer of a composition comprising a dye, a pigment, or both; and
a layer of a composition comprising an active ingredient.

2. The method of claim 1, comprising applying to the keratin substance:
a layer of at least one composition comprising, in an anhydrous medium, at least one exothermic metal salt; and
a layer of a shaving composition and/or a depilatory composition.

3. The method of claim 1, comprising applying to the keratin substance:
a layer of at least one composition comprising, in an anhydrous medium, at least one exothermic metal salt; and
a layer of a composition comprising a dye, a pigment, or both.

4. The method of claim 1, comprising applying to the keratin substance:
a layer of at least one composition comprising, in an anhydrous medium, at least one exothermic metal salt; and
a layer of a composition comprising an active ingredient.

5. The method according to claim 1, which is a cosmetic method for treating, smoothing and/or reducing wrinkles and fine lines of the skin that comprises applying to the skin of a person in need thereof a layer of a composition comprising, in an anhydrous medium, at least one exothermic metal salt.

6. The method according to claim 1, which is a cosmetic method for camouflaging and/or reducing the appearance of marks or the inhomogeneity of the skin, that comprises applying to the skin of a person in need thereof a layer of a composition comprising, in an anhydrous medium, at least one exothermic metal salt.

7. The method according to claim 1, which is a cosmetic method for treating and/or reducing cellulite, including treating and/or reducing the appearance of cellulite, that comprises applying to the skin of a person in need thereof a layer of a composition comprising, in an anhydrous medium, at least one exothermic metal salt.

8. The method according to claim 3, wherein the composition comprises at least one selected from azo dyes, pigments, and encapsulated pigments comprising a rupturable capsule.

9. The method according to claim 1, which is a method for preventing and/or reducing the adhesion of microorganisms to the skin and/or a mucous membrane, comprising the application to the skin and/or a mucous membrane of a person in need thereof of a composition comprising, in an anhydrous medium, at least one exothermic metal salt.

10. The method according to claim 1, which is a method for treating and/or reducing bags and/or dark circles under the eyes that comprises applying to the bags and/or dark circles:
a layer of a composition comprising, in an anhydrous medium, at least one exothermic metal salt; and
a layer of a composition comprising at least one active ingredient chosen from caffeine and derivatives thereof, caffeine citrate, theophylline and derivatives thereof, theobromine, acefylline, aminophylline, chloroethyltheophylline, diprofylline, diniprophylline, etamiphylline and derivatives thereof, etofylline, proxyphylline, ephedrine and derivatives thereof, combinations of caffeine and silanol, compounds of natural origin containing xanthic bases such as extracts of tea, of coffee, of guarana, of cocoa, of mate, of cola (Cola nitida), a maritime pine bark extract, manganese gluconate, an extract of Ammi visnaga such as Visnadine from Indena, lupin extract, the protein coupling of hydrolysed wheat/palmitic acid with palmitic acid, the extract of a bitter orange blossom, vitamin P and derivatives thereof, extracts of Ruscus, of horse chestnut, of ivy, of ginseng and of sweet clover, nicotinate and derivatives thereof, lysine and derivatives thereof, rutin salts, extracts of algae and mixtures thereof.

11. The method according to claim 1, wherein the exothermic metal salt is chosen from magnesium sulphate, calcium chloride and mixtures thereof.

12. The method according to claim 2, wherein the exothermic metal salt is chosen from magnesium sulphate, calcium chloride and mixtures thereof.

13. The method according to claim 3, wherein the exothermic metal salt is chosen from magnesium sulphate, calcium chloride and mixtures thereof.

14. The method of claim 1, comprising applying to the keratin substance:
a layer of at least one composition comprising, in an anhydrous medium, at least one exothermic metal salt; and
a layer of a composition comprising a monocarbonylated or polycarbonylated self-tanning agent.

15. The method according to claim 14, wherein the self-tanning agent is dihydroxyacetone (DHA).

16. The method according to claim 14, wherein the exothermic metal salt is chosen from magnesium sulphate, calcium chloride and mixtures thereof.

17. A kit, comprising:
a composition comprising, in an anhydrous medium, at least one exothermic metal salt, and, one or more of the following:
a composition comprising a monocarbonylated or polycarbonylated self-tanning agent;
a shaving composition and/or a depilatory composition;
a composition comprising a dye, a pigment, or both; and
a composition comprising an active ingredient.

18. The kit according to claim 17, which is a shaving kit comprising:
a composition comprising, in an anhydrous medium, at least one exothermic metal salt;
a shaving composition or a depilatory composition; and
a razor.

19. The kit according to claim 17, wherein the exothermic metal salt is chosen from magnesium sulphate, calcium chloride and mixtures thereof.
